(19) United States
(12) Patent Application Publication
(10) Pub. No.: US 2008/0058281 A1
(43) Pub. Date: Mar. 6, 2008

(54) Skin Lightening Compositions Comprising Vitamines and Flavonoids

(76) Inventors: Paula Rachel Yates, Eaton Bray (GB); Rebecca Louise Charles nee Newsham, Shambrook (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/547,096

(22) PCT Filed: Mar. 10, 2005
(86) PCT No.: PCT/EP05/02596

§ 371 Date: Aug. 2, 2007

(30) Foreign Application Priority Data
Mar. 30, 2004 (EP) 04251873.8

(51) Int. Cl. A61K 31/355; A61K 31/352; A61K 31/375; A61K 31/455; A61K 31/714; A61P 17/00

(52) U.S. Cl. 514/52; 514/356; 514/456; 514/458; 514/474

(57) ABSTRACT

A composition is provided comprising a flavanoid, vitamin C, vitamin E and niacin. The composition can be used as a skin lightening agent.

### FIELD OF THE INVENTION

The present invention relates to agents for skin lightening, the use of these agents for inhibiting the production of melanin in the skin of a mammal and compositions suitable for such use.

### BACKGROUND TO THE INVENTION

Some people with naturally darker skin types desire to induce a degree of lightening in their overall skin colour. Skin colour is determined primarily by the amount and type of melanin, a substance which is produced within the skin by melanocytes which reside in the epidermis. Melanin is present in two forms, namely dark melanin and light melanin. Skin lightening would result if the production of dark melanin were reduced and/or the ratio of light melanin to dark melanin production were increased.

### SUMMARY OF THE INVENTION

We have now found that a combination of a flavanoid, vitamin C, vitamin E and niacin synergistically reduces dark melanin production and enhances light melanin production.

Accordingly, the present invention provides a composition comprising a flavanoid, vitamin C, vitamin E and niacin. Preferably the flavanoid is in the form of a pine bark extract.

Further reductions in dark melanin production, and increases in light melanin production, were obtained when vitamin A was included. Accordingly, in a preferred embodiment, the composition further comprises vitamin A.

Even greater reductions in dark melanin production, and increases in light melanin production, were obtained when vitamin B12 and cysteine were included in the composition. Accordingly, in another preferred embodiment, the composition further comprises vitamin B12 and/or cysteine.

The composition can be formulated for topical and/or systemic administration. For example, the composition may be formulated as a solid dosage form or as a topical composition.

The present invention also provides a method of inhibiting the production of melanin in the skin of a mammal, the method comprising administering a composition of the invention to said mammal.

In a related aspect, the present invention further a composition of the invention for use in inhibiting the production of melanin in the skin of a mammal.

The present invention further provides a method of increasing the ratio of light melanin to dark melanin in the skin of a mammal, the method comprising administering to said mammal, a composition of the invention.

The present invention also provides a composition of the invention for use in increasing the ratio of light melanin to dark melanin in the skin of a mammal.

In another aspect the present invention a composition of the invention in the manufacture of a composition for inhibiting the production of melanin and/or increasing the ratio of light melanin to dark melanin in the skin of a mammal.

The present invention also provides a method of inhibiting in the skin of an individual, the transport of melanin from melanocytes to keratinocytes, which method comprises administering to said individual a composition of the invention.

The present invention further provides a method of inhibiting the production of melanin, such as inhibiting the production of dark melanin, in photo-protected areas of the skin of a mammal, the method comprising administering to said mammal, a composition of the invention. In one embodiment, the composition is in topical form and is administered to said photo-protected areas.

In a related aspect, the present invention further provides a method of increasing the ratio of light melanin to dark melanin in photo-protected areas of the skin of a mammal, the method comprising administering to said mammal, a composition of the invention. In one embodiment, the composition is in topical form and is administered to said photo-protected areas.

### DETAILED DESCRIPTION OF THE INVENTION

Unless defined otherwise, all technical and scientific terms used herein have the same meaning as commonly understood by one of ordinary skill in the art.

Compositions and Product Forms

Flavanoids are polyphenolic compounds and are widely found in nature. There are several classes of flavanoids: flavanols, flavonols, flavones, isoflavones, flavanones, proanthocyanidins, anthocyanidins and hydroxystilbenes. Many of these compounds exist in glycosylated forms, especially as O-glycosides. Typically, glycosylated forms are preferred over the aglycone.

Flavonols include quercitin, kaempferol and myricetin. Flavanols include catechin, epicatechin, gallocatechin, epigallocatechin, and esters thereof with gallic acid, i.e. catechin gallate epicatechin gallate, gallocatechin gallate and epigallocatechin gallate (EGCg). Flavanones include naringenin, hesperetin and sakranetin. Flavones include luteolin and apigenin. Isoflavones include daidzein and genistein. Hydroxystilbenes include resveratrol and oxyresveratrol.

The compounds can be chemically synthesised or obtained from plant materials.

A plant extract differs from the intact plant material in that the various components present in the intact plant material will be present in different amounts in the extract, or substantially absent. Prior to extraction, plant materials may be dried and or mechanically processed, e.g. crushed.

Extracts of plant materials are typically made by solvent extraction. Solvents include “solvent” includes polar and non-polar organic solvents, water, and mixtures thereof. Preferred solvents are water, ethanol and mixtures thereof. Extraction procedures may include a heating step. Solvent extracted components may be subject to further purification/separation steps such as chromatography or fractional distillation. As used herein, “fraction” means any fractioned part of a solvent containing one or more of the active ingredients described above, e.g. obtained by chromatography or by fractional distillation.

Suitable plant sources of the various polyphenolic compounds described above include fresh fruit such as grapes (skin and seeds in particular), cranberry, blackcurrants, blackberries and citrus fruits, and vegetables such as onions, kale, broccoli and French beans.

The solubility of flavonols in aqueous solvents can be increased by co-dissolving one or more anthocyanidins (see U.S. Pat. No. 6,569,446).

In a preferred embodiment, the composition comprises a mixture of proanthocyanidins and anthocyanidins. Preferably the mixture of proanthocyanidins and anthocyanidins is provided as an extract of pine bark, more preferably an extract of the bark of French maritime pine (Pinus pinatus). One such extract is available commercially as Pycnogenol™.

In another preferred embodiment, the composition comprises one or more flavonols. Preferably, the composition comprises myricetin and/or quercetin, more preferably quercetin.

In another preferred embodiment, the composition comprises one or more flavanones. Preferably, the composition comprises naringenin, hesperitin and/or sakranetin, more preferably hesperitin.

Other compounds and/or plant extracts containing the same can be tested to confirm that they possess suitable activity using, e.g, the assay methods described in the examples.

Compositions of the invention may comprise mixtures of two or more flavanoids. In one embodiment, different flavanoids are provided as different plant extracts.

Preferably the pine bark extract is an extract of the bark of French maritime pine (Pinus pinatus). One such preferred extract is available commercially as Pycnogenol™.

A preferred amount of flavanoids in a composition of the invention is at least 10 or 20 mg.

As used herein, “vitamin A” includes retinol and other chemically similar compounds referred to as retanoids. It also includes precursors such as beta-carotene and other carotenoids (provitamins) that are converted into retinol as the body requires.

A preferred amount of Vitamin A in a composition of the invention is at least 0.1 or 0.2 mg.

As used herein, “vitamin C” means ascorbic acid or the organic or inorganic (e.g. sodium) salts thereof. Mixtures of one or more of the acid and its salts are also included.

A preferred amount of Vitamin C in a composition of the invention is at least 100 or 200 mg.

As used herein, “vitamin E” includes alpha-, beta-, gamma- and delta-tocopherol in any isomeric form thereof or any mixture thereof (including mixtures of isomeric forms of any of these). Derivatives of vitamin E can be oil-soluble or water-soluble. Examples of oil-soluble vitamin E derivatives, including ester derivatised vitamin E, tocopheryl acetate, tocopheryl linoleate, tocopheryl linoleate/oleate, tocopheryl nicotinate, and tocopherol (vitamin E alcohol). Water soluble vitamin E derivatives include sodium vitamin E phosphate (VEP), lauryl imino dipropionic acid tocopheryl phosphate, tocopheryl glucoside, tocopheryl succinate, tocophersolan (tocopheryl polyethylene glycol 1000 succinate), tocophereth-5, 10, 12, 18, and 50 (polyethylene glycol (PEG) tocopheryl ethers). For the PEG vitamin E derivatives, increasing numbers represent increasing numbers of PEG molecules attached to the vitamin E. Thus, as the number increases, so does water solubility, with tocopereth-5 having the lowest water solubility and tocopereth-50 having the greatest solubility in water. Derivatives of vitamin E as referred to herein have at least 50% of the biological activity of alpha-tocopherol, for example, at least 50% of the antioxidant activity of alpha-tocopherol.

A preferred amount of vitamin E in a composition of the invention is at least 100 or 200 mg.

The term “vitamin B12” or “cobalamin” cyanocobalamin, hydroxocobalamin, and nitrocobalamin. Where the composition comprises vitamin B12, it is preferred that the composition also comprises vitamin A.

A preferred amount of vitamin B12 in a composition of the invention is at least 10 or 20 μg.

“Niacin” is the generic name for a group of compounds which exhibit niacin activity, and includes niacinamide and nicotinic acid. Preferably, niacin is provided as niacinamide.

A preferred amount of niacin, e.g. niacinamide in a composition of the invention is at least 10 or 20 mg.

As used herein, the term “cysteine” includes salts thereof. Where the composition comprises cysteine, it is preferred that the composition also comprises vitamin A.

A preferred amount of cysteine, e.g. L-cysteine in a composition of the invention is at least 50 or 100 mg.

The compositions of the present invention may be provided in forms for topical and/or systemic administration. For example, all active ingredients may be dosed systemically or all may be dosed topically or some may be dosed systemically and the remainder topically. The flavanoid component may comprise two or more different fractions or products of two or more different extraction processes. These may be all dosed systemically or all topically, or respectively split between topical and systemic delivery forms.

Thus, the term “composition” as used herein in the context of a composition according to the present invention refers both to unitary compositions containing all essential ingredients. However, the term also covers the situation where individual components of the overall composition are split between two different compositional forms which are supplied together as a product. For example, a product may comprise one compositional form for systemic delivery of its component(s) and one compositional form for topical delivery of its component(s). Examples of products containing combinations of such compositional forms are a skin cream and a nutritional supplement tablet.

Topical Compositions

In one embodiment, the compositions of the invention are formulated for topical administration, i.e. the composition is in the form of a topical composition. Accordingly, the compositions of the invention can be administered topically to a subject, i.e., by the direct laying on or spreading of the composition on skin. Such compositions can be prepared by combining a safe and effective amount of the active substance or substances as described above with a pharmaceutically-acceptable topical carrier or diluent, i.e. a dermatologically acceptable carrier or diluent.

The composition typically contains from about 0.01% to about 35% by weight of each of the active ingredients, preferably from about 0.1 wt % to about 35 wt %, more preferably from about 1 wt % to about 35 wt %, such as from 5 or 10 wt % to about 25 wt %. The total amount of active ingredients is typically from about 1 wt % to 90 wt %, more preferably at least 10 wt %.

The topical compositions useful in this invention may be made into a wide variety of product types. These include, but are not limited to lotions, creams, gels, sticks, sprays, ointments and pastes. These product types may comprise several types of carrier systems including, but not limited to solutions, emulsions, gels and solids.

The topical compositions useful in this invention formulated as solutions typically include a pharmaceutically-acceptable aqueous or organic solvent. The terms “pharmaceutically-acceptable aqueous solvent” and “pharmaceutically-acceptable organic solvent” refer to a solvent which is capable of having dispersed or dissolved therein the active(s), and possesses acceptable safety properties (e.g., irritation and sensitisation characteristics). Examples of suitable organic solvents include: propylene glycol, polyethylene glycol (200-600), polypropylene glycol (425-2025), poly vinyl pyrrolidine, propylene glycol-14 butyl ether, glycerol, 1,2,4-butanetriol, sorbitol esters, 1,2,6-hexanetriol, ethanol, isopropanol, butanediol, and mixtures thereof. These solutions preferably contain from about 0.1 wt % to about 20 wt %, more preferably from about 1 wt % to about 20 wt % more preferably still from about 1 wt % to about 10 wt %, of each active.

If the topical compositions useful in this invention are formulated as an aerosol and applied to the skin as a spray-on, a propellant is added to a solution composition.

Topical compositions may be formulated as a solution comprising an emollient, i.e. a material used for the prevention or relief of dryness, as well as for the protection of the skin. A wide variety of suitable emollients are known and may be used herein (see Sagarin, Cosmetics, Science and Technology 2nd Edition, Vol. 1, pp. 32-43 (1972)). Such compositions preferably contain from about 2% to about 50% of a topical pharmaceutically-acceptable emollient.

If the carrier is formulated as an emulsion, preferably from about 1% to about 10%, more preferably from about 2% to about 5%, of the carrier system comprises an emulsifier. Emulsifiers may be non-ionic, anionic or cationic. Suitable emulsifiers are disclosed in, for example, McCutcheon's Detergents and Emulsifiers, North American Edition, pages 317-324 (1986).

Single emulsion skin care preparations, such as lotions and creams, of the oil-in-water type and water-in-oil type are well known in the cosmetic art. Such emulsions can stabilise and enhance the penetration of actives. Multiphase emulsion compositions, such as the water-in-oil-in-water type may also be used. In general, such single or multiphase emulsions contain water, emollients and emulsifiers as essential ingredients.

Another emulsion carrier system that can be used is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other non-ionics; and from about 7% to about 20% water.

Liposomal formulations can also be used. These formulations can stabilise actives and also improve delivery of actives which do not penetrate well. Such compositions can be prepared by first combining the active with a phospholipid, such as dipalmitoylphosphatidyl choline, cholesterol and water according to the method described in Mezei & Gulasekharam, Journal of Pharmaceutics and Pharmacology, Vol. 34 (1982), pp. 473-474, or a modification thereof. Epidermal lipids of suitable composition for forming liposomes may be substituted for the phospholipid. The liposome preparation is then incorporated into one of the above topical carrier systems (for example, a gel or an oil-in-water emulsion) to produce the liposomal formulation. Other compositions and cosmetic/pharmaceutical uses of topically applied liposomes are described in Mezei, M., “Liposomes as a Skin Drug Delivery System”, Topics in Pharmaceutical Sciences (D. D. Breimer and P. Speiser, eds.), Elsevier Science Publishers B. V., New York, N.Y., 1985, pp. 345-358.

If the topical compositions are formulated as a gel or a cosmetic stick, such compositions can be formulated by the addition of a suitable amount of a thickening agent to a cream or lotion formulation.

Topical compositions may also be formulated as makeup products, such as foundations. Foundations are solution or lotion-based with appropriate amounts of thickeners, pigments and fragrance.

Various water-soluble materials may also be present in the compositions. These include humectants, proteins and polypeptides and preservatives. In addition, the topical compositions useful herein can contain conventional cosmetic adjuvants, such as dyes, opacifiers (e.g., titanium dioxide), pigments and perfumes.

The topical compositions useful in this invention may also include a safe and effective amount of a penetration enhancing agent. A preferred amount of penetration enhancing agent is from about 1% to about 5% of the composition. Examples of useful penetration enhancers are described in U.S. Pat. No. 6,068,834. Other conventional skin care product additives may also be included in the compositions. For example, collagen, hyaluronic acid, elastin, hydrolysates, primrose oil, jojoba oil, epidermal growth factor, soybean saponins, mucopolysaccharides, and mixtures thereof may be used.

It may be desirable to include in the compositions of the invention, one or more sun screening agents. A wide variety of conventional sun screening agents are disclosed in, for example, Cosmetics, Science and Technology 2nd Edition (1972), Vol. 1, Chapter VIII, pages 189 et seq. See also U.S. Pat. No. 6,068,834.

The sun screening agent must be compatible with the active(s). The composition preferably comprises from about 1% to about 20%, more preferably from about 2% to about 10%, of a sun screening agent. Exact amounts will vary depending upon the sunscreen chosen and the desired Sun Protection Factor (SPF).

An agent may also be added to any of the compositions of the invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157.

The present invention relates to methods of inhibiting melanin production in the skin of a mammal, typically a human. In one embodiment, such methods comprise the administration of a safe and effective amount of a composition of the invention to the skin or regions thereof the skin. The amount of active agent and frequency of application will vary depending on the initial condition of the skin and the desired end result. Generally, the compositions should be administered in a sufficient amount and for a sufficient period of time to visibly whiten the skin.

Any dose which is less than the toxic level may be used, thus it is contemplated that for certain dosage forms, particularly topical dosage forms, the “dose” is any amount that provides the desired effect, and that amount may be so large due to frequency of application and amount applied that the maximum effective amount is irrelevant.

A safe and effective amount of active in a topical composition is applied, generally from about 1 μg to about 1 mg per cm² skin per application, preferably from about 2 μg to about 800 μg/cm² skin per application, more preferably from about 30 μg to about 700 μg/cm² skin, most preferably from about 75 μg to about 250 μg/cm² skin. Frequency of application typically ranges from about four times a day to about twice a week, more preferably from about three times a day to about once every other day, more preferably at least twice daily. It is generally preferred that at least one application occurs in the evening.

Systemic Compositions

Compositions of the invention can be combined with a pharmaceutically acceptable carrier or diluent to produce a pharmaceutical composition. Pharmaceutically acceptable diluents or carriers suitable for use in such compositions are well known in the art of pharmacy. The compositions of the invention typically contain from 0.1 to 35% by weight of each active, such as from 1 to 25% by weight of active, more preferably at least 5 or 10 wt % of active.

The pharmaceutical composition may consist of solid dosage forms such as tablets, hard gelatin capsules, soft gelatin capsules, bulk powders, and microcapsules of the drug. Alternately, it may consist of a liquid dosage form such as an aqueous or nonaqueous solution, emulsion, or suspension.

Solid compositions for oral administration are preferred compositions of the invention. Solid compositions of the invention are preferably prepared in unit dosage form, such as in the form of tablets and capsules. Suitably tablets may be prepared by mixing the active combination with an inert diluent such as calcium phosphate in the presence of disintegrating agents, for example maize starch, and lubricating agents, for example magnesium stearate, and tableting the mixture by known methods. Such tablets may, if desired, be provided with enteric coatings by known methods, for example by the use of cellulose acetate phthalate. Similarly, capsules, for example hard or soft gelatin capsules, containing the active combination optionally in the form of beads with or without added excipients, may be prepared by conventional means and, if desired, provided with enteric coatings in a known manner. The tablets may be formulated in a manner known to those skilled in the art so as to give a controlled release of the compound of the present invention.

Controlled release forms of the pharmaceutical compositions of the present invention include rapid release formulations such as soluble granules or melt filled fast release capsules, delayed release formulations such as tablets provided with enteric coatings, for example, of cellulose acetate phthalate and, in particular, sustained release formulations. Numerous types of sustained release formulations are known to those skilled in the art. Typically, the active combination may be encapsulated within a release retarding coating, for example, a copolymer of cellulose ether and acrylate, or may be bound to small particles such as, for example, ion exchange resin beads. Alternatively, the active combination may be incorporated into a matrix containing a release retarding agent such as a hydrophilic gum e.g. xanthan gum, a cellulose derivative eg. hydroxypropyl methylcellulose, or a polysaccharide, wax or plastics material.

The active combination may be formulated into a solid dosage form in which the two active ingredients are kept separate. For example, the dosage form may be a bilayer tablet in which the active ingredients are contained in different layers. The different layers can be formulated so as to provide the optimum release profile for each drug.

Liquid fill compositions for example viscous liquid fills, liquid paste fills or thixotropic liquid fills are also suitable for oral administration. Melt filled compositions may be obtained by mixing the active combination with certain esters of natural vegetable oil fatty acids, for example, the Gelucire™ range available from Gattefosse to provide a variety of release rates. Suitably a melt-filled capsule comprises from 10 to 80% total active and from 20 to 90% of a fatty acid ester excipient which comprises one or more polyol esters and triglycerides of natural vegetable oil fatty acids.

Preferably oral liquid compositions comprise from 1 to 5 wt % of each active together with from 1 to 50 wt % of a diluent, the remainder made up with sterile water. Optionally the composition may contain suspending agents, thickeners, cosolvents such as alcohol and/or preservatives. Suitable diluents include sweetening agents for example sorbitol, xylitol or sucrose. Suitable suspending agents or thickeners include cellulose gums, agar or natural gums, for example xanthan gum. Flavourings or other taste-masking agents known to those skilled in the art for example saccharin, sodium saccharin, acesulpham K or aspartame may be added.

Compositions of the invention suitable for parenteral administration can be prepared by combination of the active with known pharmaceutical forms for such administration, for example sterile suspensions or sterile solutions of the active in a suitable solvent such as saline.

The preferred mode of administration is orally.

Generally, the compositions should be administered in a sufficient amount and for a sufficient period of time to visibly whiten the skin.

The amount of the compound administered depends upon the bioavailability of the compound from the pharmaceutical composition, in particular where oral administration is used. Typically, however, the compounds of this invention are dosed in an amount of from about 0.01 mg/kg of body weight to about 100 mg/kg, preferably from about 0.1 to about 30 mg/kg of body weight. The amount of the pharmaceutical composition depends upon the percent of compound within its formula, which is a function of the amount of the compound required per dose, its stability, release characteristics and other pharmaceutical parameters. The doses are typically administered from once or twice weekly to one or twice daily.

Preferred dosages of actives are set out, independently, below:

Flavanoids, e.g. Pycnogenol: at least 20 or 50 mg/day.

Vitamin C: at least 200 or 500 mg/day.

Vitamin E: at least 500 mg/day.

Vitamin A at least 0.5 or 1 mg/day.

Niacin, e.g. niacinamide: at least 20 or 50 mg/day.

Vitamin B12 at least 20 or 50 μg/day.

L-cysteine at least 100 or 200 mg/day

The routes of administration and dosages described are intended only as a guide since a skilled practitioner will be able to determine readily the optimum route of administration and dosage for any particular individual.

Another means of systemic dosing comprises dosing any of the aforementioned compositions in a food product which does not therefore necessarily require use of a pharmacologically acceptable carrier.

As used herein, the term “food products” includes both food products as such and beverages. Suitable food products as such include spreads, dairy products (including milk and yoghurts), desserts, convenience foods/snacks, breakfast cereals and cereal bars, ready-cook meals, bread and frozen confections such as ice creams, water ices and sorbets and yoghurt ice creams. Food products also include dietary/nutritional supplements. Suitable beverages include tea, tea-flavoured drinks, coffee, soft drinks (e.g. carbonated squashes etc) and fruit juice.

The food products are typically supplemented with the active ingredients of the invention so that they contain higher amounts of the active ingredient(s) than they would normally contain.

Where the active ingredients are split between topical and systemic regimes, the upper and lower values of the optimum daily dosage ranges are proportioned according to the split between topical and systemic as appropriate.

Uses

The compositions of the invention can be used to modulate melanin production in the skin of a mammal, in particular a human. More specifically, they can be used to increase the ratio of light melanin:dark melanin in skin, for example by inhibiting the production of dark melanin (eumelanin) in skin. Preferably the production of dark melanin is reduced by at least 15%, more preferably at least 20% or 25%. The production of light melanin may be increased, decreased or remain substantially unchanged. Preferably the production of light melanin is increased by at least 20%, more preferably by at least 50%. Changes in melanin production can be determined, for example, using the Melanoderm™ system as described below in the experimental section.

Preferably the ratio of light melanin:dark melanin is increased at least 1.5-fold relative to the control (measured as the percentage of light melanin relative to the control divided by the percentage of dark melanin relative to the control e.g. if light melanin is increased to 150% of the control and dark melanin is decreased to 50% of the control, the ratio is 3:1 relative to the control). More preferably the ratio of light melanin:dark melanin is increased at least 2-fold. Consequently, the compositions of the invention can be used to induce skin lightening in mammals such as humans. The advantage of increasing the ratio of light melanin:dark melanin in skin rather than simply inhibiting production of both types of melanin is that a better skin tone is produced.

In one embodiment, the compositions are used to induce skin lightening, such as inhibit dark melanin production and/or increase the ratio of light melanin:dark melanin, in photo-protected/sun-protected areas of skin of an individual.

The present invention will now be described further with reference to the following examples which are illustrative only and non-limiting.

### EXAMPLE 1

In these examples, evaluation of the ability of various agents, individually and in combination, to influence levels of dark and light melanin was tested using the commercially available Melanoderm™ system.

The source of flavanoids was pine bark extract (Pycnogenol), obtained from Solgar (Pycnogenol® 30 mg). Other agents were obtained from Sigma.

TABLE 1
agents tested.

| Compound | Supplier | Concentration |
|---|---|---|
| Pycnogenol | Solgar (Pycnogenol ® | 10 μg/ml |
| | 30 mg) | |
| Vitamin C | Sigma | 30 μg/ml |
| Vitamin E | Sigma | 1 μM |
| Vitamin B12 | Sigma | 1 ng/ml |
| Vitamin A | Sigma | 15 ng/ml |
| Cysteine | Sigma | 10 μg/ml |
| Niacinamide | Sigma | 10 μg/ml |

Combinations Tested:
1. Niacinamide
2. Vitamin A
3. Vitamin B12
4. Cysteine
5. Pycnogenol+Vitamin C+Vitamin E
6. Pycnogenol+Vitamin C+Vitamin E+Niacinamide
7. Pycnogenol+Vitamin C+Vitamin E+Niacinamide+Vitamin A
8. Pycnogenol+Vitamin C+Vitamin E+Niacinamide+Vitamin A+Vitamin B12+Cysteine.
   Treatment Regime for Melanoderms™

The MelanoDerm™ MatTeks system consists of normal, human-derived epidermal keratinocytes (NHEK) and melanocytes (NHM) which have been cultured to form a multilayered, highly differentiated model of the human epidermis. The NHM within co-cultures undergo spontaneous melanogenesis leading to tissues of varying levels of pigmentation. The tissues are produced using serum free medium without artificial stimulators of melanogenesis such as TPA and IBMX. The cultures are grown on cell culture inserts at the air-liquid interface, allowing for simulated topical application of agents to be tested. Introduction of agents into the medium simulates systemic application. Thus, the model provides a useful in vitro means to evaluate agents designed to modulate skin pigmentation.

On delivery, the melanoderms (MatTek MEL-300-B) were placed onto metal ring supports in a 6 well plate containing 5 ml of pre-warmed maintenance media (of EPI-100-MM-PRF), using aseptic technique as per MatTek's protocol. Incubation was carried out overnight at 37° C. and 4% CO₂ to allow the melanoderms to recover and equilibrate fully. Once placed under these conditions the MEL-300 tissue, undergo melanogenesis and differentiation.

Treatment was initiated on the following morning. Agents to be tested were dissolved in appropriate solvents and added to warmed media at final concentrations pre-assessed for melanocyte toxicity. Each time the media was changed, the spent media was aspirated from the melanoderms and reserved for testing for toxicity (Lactate Dehydrogenase (Promega)) and Interleukin-1 release (R&D systems) and replaced with a fresh dose of media plus test agents whether within the media of the melanoderm. Melanoderms were returned to the incubator. This treatment regime was repeated every 48 hours until a relative difference in darkening was observed between control and test agents.

On observation of differences in darkening, microscopic and macroscopic darkening were recorded photographically. Harvesting the tissue of the melanoderm involved cutting away the tissue from its plastic support and was followed by quantification of melanin present post-treatment versus untreated.

Selective Solubilisation of Melanin from Melanoderm Tissue

(1) Quantification of Alkali-Soluble Melanin (Light Melanin)

Melanoderm samples were cut from the plastic holders and the wet weight of tissue measured. 200 μl 1M NaOH/8M urea was added to the melanoderm sample the tissue homogenised in a microfuge tube. Samples were whirlimixed at RT for 30 minutes on and off to release the soluble melanin. Samples were centrifuged at 13,000 rpm for 10 minutes and supernatant containing soluble melanin was removed to a fresh tube.

Protein was extracted from the supernatant by addition of 200 μl chloroform and then by mixing vigorously for 1 minute. Phases were separated by centrifugation at 13,000 rpm for 10 minutes. 150 μl of supernatant was added to a microtitre plate (in duplicate) and the OD 340 nM ascertained.

(2) Quantification of Alkali-Insoluble Melanin (Dark Melanin)

1M NaOH was added to the remaining pellet which contains the insoluble melanin and the sample vortexed for one minute. The sample was then incubated in a water bath at 37° C. for 96 hrs with daily mixing to released the insoluble melanin. The sample was centrifuged for 10 minutes at 13,000 rpm with 200 μl of chloroform and 190 μl of the supernatant taken to a fresh tube. The supernatant was centrifuged again and 150 μl removed to a microtitre plate for analysis of absorption at 340 nm.

Calculation of Absolute Melanin Concentration

Absolute melanin is calculated as the actual melanin quantity calculated from a previously determined light melanin standard curve:
x=(y−0.003)/4.76423 (where x=concentration of melanin and y=optical density at 340 nm).

For dark melanin, the curve is x=(y−0.00553)/3.70312.

Results

| | | | | Ratio of light |
|---|---|---|---|---|
| | Total | Light | Dark | melanin (μg/g)/ |
| | Melanin | Melanin | Melanin | dark melanin |
| | μg/g | μg/g | μg/g | (μg/g) |
| Control | 6.93 | 0.85 | 6.08 | 0.14 |
| Niacinamide | 8.10 | 0.94 | 7.16 | 0.13 |
| | | (+10.6%) | (+17.8%) | |
| Vitamin A | 7.81 | 1.09 | 6.71 | 0.16 |
| | | (+28.2%) | (+10.4%) | |
| Vitamin B12 | 7.93 | 1.27 | 6.66 | 0.19 |
| | | (+49.4%) | (+9.5%) | |
| Cysteine | 5.96 | 0.69 | 5.27 | 0.13 |
| | | (−18.8%) | (−13.3%) | |
| Pyc + Vit C + Vit E | 8.83 | 1.15 | 7.69 | 0.15 |
| | | (+35.3%) | (+26.4%) | |
| Pyc + Vit C + Vit E + | 6.18 | 1.07 | 5.11 | 0.21 |
| Niacinamide | | (+25.9%) | (−16.0%) | |
| Pyc + Vit C + Vit E + | 5.92 | 1.43 | 4.49 | 0.32 |
| Niacinamide + Vit A | | (+68.2%) | (−26.2%) | |
| Pyc + Vit C + Vit E + | 5.77 | 1.65 | 4.12 | 0.40 |
| Niacinamide + Vit A + | | (+94%) | (−32.2%) | |
| Vit B12 + Cysteine | | | | |

Treatment with a combination of Pycnogenol+vitamin C+vitamin E+niacinamide reduced the total melanin produced by the melanoderm to a much greater extent than either niacinamide alone or a combination of Pycnogenol+vitamin C+vitamin E. Further inhibition of melanin production was seen when vitamin A was added for produce a five-component composition. This reduction was further enhanced by the inclusion of vitamin B12 and cysteine. Soluble melanin was increased in all three cases and insoluble melanin was decreased in all three cases.

These results indicate that a combination of a flavanoid (Pycnogenol), vitamin C, vitamin E and niacinamide inhibits dark melanin production in a synergistic manner and increases light melanin production in all cases. Further effects were seen in combination with vitamin A with the best results being obtained with a seven component combination also including vitamin B12 and cysteine.

### EXAMPLE 2

### In Vivo Efficacy: Human Clinical Study

Study Objectives

There were two primary objectives for this study:

To examine the hypopigmenting effects of an oral nutritional supplement.

To examine the possible synergistic skin lightening effects between an oral nutritional supplement and topical lightening product.

Design and Methods

The study was a parallel, double blind, randomised, placebo-controlled exploratory trial. 80 participants were selected to participate in the study based on sub-optimal micronutrient status. They were randomised in a parallel design into two groups of equal size to received supplements with or without the active ingredients for a period of 12 weeks. During the intervention period subjects also applied a topical product to one arm. The topical lightening product was the commercially available Ponds Double White. Subjects acted as their own control when investigating the synergy between oral and topical skin lightening.

Photography, chromameter, expert assessment, Mexameter, directed difference, blood and urine sampling were performed before the intervention (baseline, T1), after 4 weeks of the intervention (T5), after 8 weeks of the intervention (T9) and again at the end of the intervention (T13). A self-assessment questionnaire concerning self-perceived change in skin colour was also completed at these times. Sampling to assess the vitamin status in skin and sebum collection was performed before the intervention (baseline, T1), after 8 weeks of the intervention (T9) and again at the end of the intervention (T13). Hydration and barrier function were performed at baseline (T1), after 8 weeks of the intervention (T9) and at the end of the intervention (T13). Elasticity measurements, replica collection and skin biopsies were performed at baseline (T1) and at the end of the intervention (T13).

Subjects were healthy, female volunteers aged between 20-50 yrs old, had a sub-optimal micronutrient status, a BMI of 18-23 kg/m² and were non-smokers.

Oral Product

Subjects were provided with capsules (product/placebo). One dosage of the oral supplement consisted of 3 capsules, i.e. one fat-soluble and two water-soluble capsules. Each person consumed 2 dosages per day.

Those subjects receiving the test capsules consumed:
Pycnogenol 100 mg/day
Vitamin C 1000 mg/day
Vitamin E 500 mg/day
Vitamin A 1.5 mg/day
Niacinamide 100 mg/day
Vitamin B12 100 μg/day
L-cysteine 500 mg/day
   Topical Product

A measured amount of product was applied to the treatment arm from shoulder to wrist twice daily. Ponds Day Essence was applied in the morning and Ponds Night Essence at night (after taking a bath or shower).

The primary clinical endpoint was skin lightness at a “sun-protected” site on the inner upper (IU) arm at T13, as measured by chromameter. This calorimetric method measures overall lightness/darkness of skin by recording white-black (L), green-red (a) and yellow-blue (b) colour spectrum from light reflected by skin.

Results

Sixty-eight female Japanese subjects completed the study.

Supplements prevented skin darkening of inner-upper arm compared to placebo when measured with chromameter. The difference between groups was statistically significant when comparing white-black (L), green-red (a) and yellow-blue (b) color spectra reflected by skin (P=0.045, P=0.038 and P=0.031, respectively).

Supplements improved the crow foot area when compared with placebo. The difference was statistically significant (P=0.01).

Supplements prevented skin darkening of inner-upper arm at least as good or better than Ponds Double White.

The various features and embodiments of the present invention, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

1. A composition comprising a flavanoid, vitamin C, vitamin E and niacin.

2. A composition according to claim 1, further comprising vitamin A.

3. A composition according to claim 2, further comprising vitamin B12.

4. A composition according to claim 2 or claim 3, further comprising cysteine.

5. A composition according to any one of claims 1 to 4 wherein the flavanoid is present in the form of a pine bark extract.

6. A composition according to any one of the preceding claims wherein the composition is formulated for systemic administration.

7. A composition according to any one of claims 1 to 6, wherein the composition is formulated for topical administration.

8. A skin lightening product comprising a solid dosage form comprising an effective amount of a composition according to any one of the preceding claims.

9. A topical skin lightening product an effective amount of a composition according to any one of claims 1 to 7.

10. A method of inhibiting the production of melanin in the skin of a mammal, the method comprising administering to said human an effective amount of a composition according to any one of claims 1 to 7.

11. A method according to claim 10 wherein the composition is administered systemically.

12. A method according to claim 10 wherein the composition is administered topically.

13. A composition according to any one of claims 1 to 7 for use in inhibiting the production of melanin in the skin of a mammal.

14. Use of a composition according to any one of claims 1 to 7 in the manufacture of a composition for inhibiting the production of melanin in the skin of a mammal.

15. A method of increasing the ratio of light melanin to dark melanin in the skin of a mammal, the method comprising administering to said human an effective amount of a composition according to any one of claims 1 to 7.

16. A composition according to any one of claims 1 to 7 for use in increasing the ratio of light melanin to dark melanin in the skin of a mammal.

17. Use of a composition according to any one of claims 1 to 7 in the manufacture of a composition for increasing the ratio of light melanin to dark melanin in the skin of a mammal.

18. A method of inhibiting in the skin of an individual, the transport of melanin from melanocytes to keratinocytes, which method comprises administering to said individual an effective amount a composition according to any one of claims 1 to 6.
